(19) United States
(12) Patent Application Publication
(10) Pub. No.: US 2023/0077694 A1
(43) Pub. Date: Mar. 16, 2023

(54) STRESS ESTIMATION APPARATUS, STRESS ESTIMATION METHOD, AND COMPUTER READABLE RECORDING MEDIUM

(71) Applicant: NEC Corporation, Minato-ku, Tokyo (JP)

(72) Inventors: Yoshiki NAKASHIMA, Tokyo (JP); Masanori Tsujikawa, Tokyo (JP); Terumi Umematsu, Tokyo (JP)

(73) Assignee: NEC Corporation, Minato-ku, Tokyo (JP)

(21) Appl. No.: 17/797,744

(22) PCT Filed: Feb. 14, 2020
(86) PCT No.: PCT/JP2020/005877

§ 371 Date: Aug. 5, 2022

(51) Int. Cl. A61B 5/16; A61B 5/11; A61B 5/00

(52) U.S. Cl. CPC A61B 5/165; A61B 5/1118; A61B 5/7275

(57) ABSTRACT

A stress estimation apparatus that estimates stress of a measurement subject, obtains a body-movement data acquisition unit 101 for acquiring body-movement data, a body-movement data storage unit 102 for storing body-movement data, a body-movement feature calculation unit 103 for calculating a stress-related body-movement feature from the stored body-movement data, a going-out frequency calculation unit 104 for calculating an estimated value of a going-out frequency from the stored body-movement data, a body-movement feature correction unit 105 for correcting a correlation of the body-movement feature with stress using the body-movement feature and the going-out frequency, a corrected body-movement feature output unit 106 for outputting the corrected body-movement feature, and a stress estimation unit 107 for estimating stress using the corrected body-movement feature.

### TECHNICAL FIELD

The invention relates to a stress estimation apparatus, a stress estimation method, and a computer readable recording medium.

### BACKGROUND ART

Recent years have seen the emergence of a problem in which people's mental health is damaged due to their autonomous nerves being disturbed as a result of a state in which their sympathetic nerves are active persisting over a long time as a result of long-term exposure to stressors, etc. Thus, a proposal has been made of a technique of having a measurement subject wear a wearable terminal on a daily basis to measure a biosignal reflecting bio-information (sweat rate, skin surface temperature, body movement, or the like) of the measurement subject from the wearable terminal over a long period and monitor long-term stress (chronic stress) of the measurement subject. In such a technique, it is generally necessary to estimate the activity state of the measurement subject's body using an accelerometer signal or the like in order to identify whether changes in the biosignal (sweat rate, skin surface temperature, body movement, or the like) being measured are caused by physical activities such as hard exercise or mental activities such as stress. As examples of activity states of the body (hereinafter “activity state”), a seated state (sitting), a walking state (walking), a running state (running), etc., can be mentioned, for example.

Non-Patent Document 1 discloses a technique in which, from data for 20 people over a 30-day period, three activity states (sitting, walking, and running) are identified from activity magnitude (moving average of the change in the rooted mean square (RMS) of acceleration in three axes) that is in common between all participants. In the technique disclosed in Non-Patent Document 1, next, using stress values quantified through a stress questionnaire as correct labels, stress estimation is performed using machine learning by calculating an average, a variance, a median value, a power spectrum density histogram component, etc., of sweating and body movement as features, individually for each of the three estimated activity states (sitting, walking, and running). Furthermore, Non-Patent Document 2 discloses a technique in which thresholds for distinguishing the three activity states of sitting, walking, and running from one another are automatically derived and applied, and the thresholds are automatically derived for each individual from an activity magnitude histogram of the individual. By using such methods, both Non-Patent Documents 1 and 2 enable a perceived stress scale of measurement subjects as disclosed in Non-Patent Document 3 to be estimated with a certain level of accuracy.

### LIST OF RELATED ART DOCUMENTS

### Non-Patent Document

Non-Patent Document 1: A. Sano, “Measuring College Students' Sleep, Stress, Mental Health and Wellbeing with Wearable Sensors and Mobile Phones”, Massachusetts Institute of Technology, 2015

Non-Patent Document 2: Y. Nakashima et al., “An Effectiveness Comparison between the Use of Activity State Data and That of Activity Magnitude Data in Chronic Stress Recognition”, ACII workshop, 2019

Non-Patent Document 3: S. Cohen, R. C. Kessler, and L. U. Gordon, “Measuring Stress: A Guide for Health and Social Scientists”, Oxford University Press, 1997

### SUMMARY OF INVENTION

### Problems to be Solved by the Invention

Incidentally, a body-movement signal is used as a stress feature in Non-Patent document 1. The body movement of a measurement subject can be used as an indicator of stress responses and stressors. However, while a body-movement signal is highly correlated with stress and serves as a good feature in stress estimation for measurement subjects frequently performing physical activities such as going out, the correlation of a body-movement signal with stress is low for measurement subjects who do not frequently perform physical activities such as going out. There was a problem that, due to the correlation with stress differing between a group having a high going-out frequency and a group having a low going-out frequency, stress scores could not be estimated altogether for both groups using a single model.

Note that, in the following, a “stress score” is a score that is calculated from responses to a psychological questionnaire, etc., and that reflects the psychological stress level of the respondent, and the higher the score is, the greater the stress is.

The above-described situation can be briefly explained using a conceptual diagram as illustrated in FIG. 1. In FIG. 1, in regard to a group of measurement subjects who frequently perform physical activities such as going out (hereinafter “group 1”), a certain proportional relationship can be observed between a feature for stress estimation calculated from a body-movement signal (hereinafter “body-movement feature”) and stress scores, as can be seen for example from the fact that the higher the body-movement feature is, the higher the stress scores are. Note that body-movement features in the present application are each designed so that stress is great when the feature is great. That is, it is assumed for example that, for a body-movement feature that would be inversely-proportional to stress scores if calculated without any further processing, an adjustment is performed by performing a calculation operation such as that in which a negative coefficient is multiplied therewith.

In contrast, in regard to a group of measurement subjects who do not frequently perform physical activities such as going out (hereinafter “group 2”), the correlation between the body-movement feature and stress scores is low, and there is substantially no relation between the body-movement feature and stress scores.

FIG. 1 is a schematic diagram for explaining the problem to be solved by the invention. In FIG. 1, the relationships (models) between stress scores and the body-movement feature are illustrated in a simplified manner using dotted lines. Model 1 corresponds to group 1, and model 2 corresponds to group 2. The problem is that, as illustrated in FIG. 1, the model differs depending on group, and it is difficult to use a single model for representation.

An example object of the invention is to provide a technique for estimating stress using the same model regardless of a going-out frequency so that the mental state such as stress of a measurement subject can be monitored from a biosignal on a daily basis.

### Means for Solving the Problems

In order to achieve the aforementioned object, a stress estimation apparatus according to an example aspect of the present invention is a stress estimation apparatus that estimates stress of a measurement subject, comprising:

a body-movement data acquisition unit for acquiring body-movement data;

a body-movement data storage unit for storing body-movement data;

a body-movement feature calculation unit for calculating a stress-related body-movement feature from the stored body-movement data;

a going-out frequency calculation unit for calculating an estimated value of a going-out frequency from the stored body-movement data;

a body-movement feature correction unit for correcting a correlation of the body-movement feature with stress using the body-movement feature and the going-out frequency;

a corrected body-movement feature output unit for outputting the corrected body-movement feature; and

a stress estimation unit for estimating stress using the corrected body-movement feature.

In order to achieve the aforementioned object, a stress estimation method according to an example aspect of the present invention is a stress estimation method for estimating stress of a measurement subject, comprising:

a step of acquiring body-movement data;

a step of storing body-movement data;

a step of calculating a stress-related body-movement feature from the stored body-movement data;

a step of calculating an estimated value of a going-out frequency from the stored body-movement data;

a step of correcting a correlation of the body-movement feature with stress using the body-movement feature and the going-out frequency;

a step of outputting the corrected body-movement feature; and

a step of estimating stress using the corrected body-movement feature.

In order to achieve the aforementioned object, a computer readable recording medium according to an example aspect of the present invention is a computer readable recording medium that includes recorded thereon a program including instructions that cause a computer to estimate stress of a measurement subject, the program including instructions that cause the computer to:

a step of acquiring body-movement data;

a step of storing body-movement data;

a step of calculating a stress-related body-movement feature from the stored body-movement data;

a step of calculating an estimated value of a going-out frequency from the stored body-movement data;

a step of correcting a correlation of the body-movement feature with stress using the body-movement feature and the going-out frequency;

a step of outputting the corrected body-movement feature; and

a step of estimating stress using the corrected body-movement feature.

### Advantageous Effects of the Invention

It can be assumed that the “degree of contribution” of a body-movement feature to stress would be lower for people having a lower going-out frequency. Thus, by correcting this low “degree of contribution” using the low going-out frequency, the ability of generalizing the accuracy of stress estimation between a group of people having a high going-out frequency and a group of people having a low going-out frequency would improve. Here, the “degree of contribution” refers to the proportional coefficient Cⱼᵢ when Sᵢ is the stress of an i^{th} measurement subject, and BFⱼᵢ is a j^{th} body-movement feature of the i^{th} measurement subject, as indicated in formula (1) below.

Sᵢ=CⱼᵢBFⱼᵢ [Math. 1]

Cⱼᵢ is substantially constant (the variation thereof is small) in group 1. However, in group 2, Cⱼᵢ is not constant and tends to decrease as the stress score increases, and the variation thereof is great. However, the going-out frequency tends to be low if the body-movement feature is small relative to the stress score (the “degree of contribution” of the body-movement feature is small). Thus, by performing a correction by performing multiplication with the reciprocal of the going-out frequency, etc., all data points are positioned close to group 1 (since the going-out frequency is high in group 1 in the first place, not much of a change occurs even if the correction is performed), and accurate analysis can be performed using a single model (model 1). This situation means that stress scores for the two groups can be estimated using one accurate model (dotted line), as illustrated in FIG. 2. FIG. 2 is a schematic diagram for explaining the effect of the invention.

### BRIEF DESCRIPTION OF THE DRAWINGS

FIG. 1 is a schematic diagram for explaining the problem to be solved by the invention.

FIG. 2 is a schematic diagram for explaining the effect of the invention.

FIG. 3. is a block diagram illustrating the configuration of a stress estimation apparatus in the present example embodiment.

FIG. 4 is a diagram for explaining why the stress feature can be corrected.

FIG. 5 is a diagram illustrating how stress is related with the going-out frequency and the body-movement feature.

FIG. 6 is a diagram illustrating how stress is related with the going-out frequency and the body-movement feature.

FIG. 7 is a diagram in which, in place of the going-out frequency in FIG. 5, the reciprocal of the going-out frequency is illustrated.

FIG. 8 is a diagram in which, in place of the going-out frequency in FIG. 6, the reciprocal of the going-out frequency is illustrated.

FIG. 9 is a diagram for explaining the operation in formula (8) in further detail.

FIG. 10 is a diagram for explaining the operation in formula (8) in further detail.

FIG. 11 is a diagram illustrating the operation performed to improve the accuracy of the model for estimating stress scores.

FIG. 12 is a flowchart illustrating operations of the stress estimation apparatus.

FIG. 13 is a diagram for describing a specific example of the present example embodiment.

FIG. 14 is a diagram for describing a specific example of the present example embodiment.

FIG. 15 is a diagram illustrating the correlation of a body-movement feature before and after correction with PSS.

FIG. 16 is a diagram illustrating the relation between the correction term and the body-movement feature.

FIG. 17 is a graph in which the schematic graph illustrated in FIG. 7 is verified with real data.

FIG. 18 is a graph in which the schematic graph illustrated in FIG. 8 is verified with real data.

FIG. 19 is a block diagram showing one example of a computer that realizes the stress estimation apparatus in the example embodiment.

### EXAMPLE EMBODIMENT

In the following, a configuration of one example embodiment of the invention will be described.

[Apparatus Configuration]

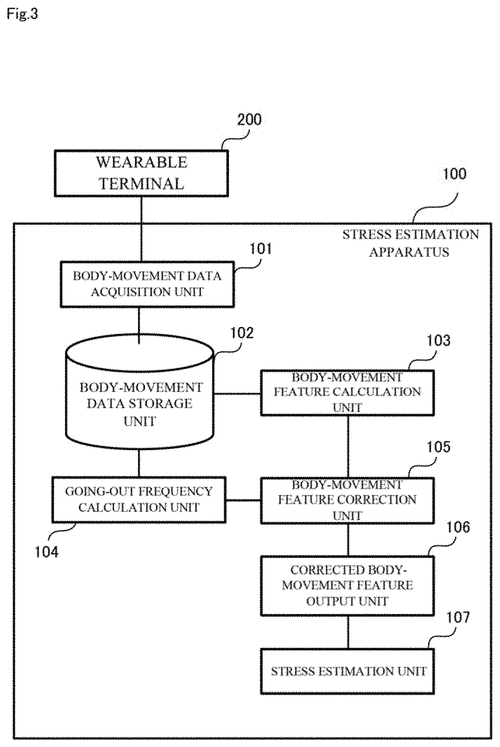

FIG. 3. is a block diagram illustrating the configuration of a stress estimation apparatus 100 in the present example embodiment.

The stress estimation apparatus 100 is an apparatus that estimates stress of a measurement subject. The stress estimation apparatus 100 can perform wired or wireless data communication with a wearable terminal 200 worn on a part of the measurement subject's body (e.g., a measurement subject's arm). Note that the wearable terminal 200 may perform data communication with a portable device terminal (smartphone or the like) owned by the measurement subject, and the stress estimation apparatus 100 and the wearable terminal 200 may perform data communication with one another via the portable device terminal.

The wearable terminal 200 measures a body-movement signal of the measurement subject. The body-movement signal is a signal that reflects the body movement of the measurement subject. An acceleration sensor signal and a gyrosensor signal can be mentioned as examples of the body-movement signal. However, the body-movement signal is not limited to such signals, and may be any signal that reflects the body movement of the measurement subject. In addition, the wearable terminal 200 may acquire bio-information other than the body-movement signal. The measurement subject's sweat rate, skin surface temperature, pulse rate, heart rate, respiratory rate, brain waves, etc., can be mentioned as examples of the bio-information other than the body-movement signal. However, there is no limitation to such information, and any information from which the mental state such as stress of the measurement subject can be estimated, such as information reflecting the autonomic nerve activity of the measurement subject, is included within the scope of the invention. Furthermore, the wearable terminal may have any shape which allows the body-movement signal alone, or the body-movement signal and a biosignal other than the body-movement signal that reflects the mental state such as stress of the subject to be measured, and which allows the wearable terminal to be worn by the measurement subject, such shapes including, besides the wrist-band-type shape such as that disclosed in Non-Patent Document 1, a badge-type shape, an ID-card-type shape, an earphone-type shape, a shirt-type shape, a head-mounted-type shape, an eyeglass-type shape, etc.

The stress estimation apparatus 100 includes a body-movement data acquisition unit 101, a body-movement data storage unit 102, a body-movement feature calculation unit 103, a going-out frequency calculation unit 104, a body-movement feature correction unit 105, a corrected body-movement feature output unit 106, and a stress estimation unit 107.

The body-movement data acquisition unit 101 acquires body-movement data from the wearable terminal 200. For example, the body-movement data is an acceleration signal detected by the wearable terminal 200.

The body-movement data storage unit 102 stores the body-movement data acquired by the body-movement data acquisition unit 101.

The body-movement feature calculation unit 103 calculates a stress-related body-movement feature (stress feature) from the body-movement data stored in the body-movement data storage unit 102. As the stress feature, an average value, a variance value, a time-series histogram, a power spectral density histogram, etc., are suitably used, as disclosed in Non-Patent Document 1 and Non-Patent Document 2. However, the stress feature is not limited to such features, as long as the stress feature is a stress-related feature derived based on the body-movement data.

Here, when a feature such as an average value, a variance value, a time-series histogram, or a power spectral density histogram is to be calculated from signals for a plurality of measurement periods with different lengths, features need to be adjusted in accordance with the lengths of the measurement periods. For example, suppose that the average value of body movement during day 1, 2, and 3 of a 3-day period was 0.4 G, 0.5 G, and 0.3 G, respectively, and the data acquisition period during day 1, 2, and 3 was 6 hours, 7 hours, and 8 hours, respectively. The average value in such a case should be calculated in a manner similar to that when a weighted average (expected value) is calculated, as 0.4*6/(6+7+8)+0.5*7(6+7+8)+0.3*8/(6+7+8). Mathematical formula (2) below schematically indicates the approach of this calculation method.

$$\begin{matrix}[ Math . 2 ] & \\ {BF}_{ji} = {\sum }_{k = 1}^{n} \frac{{l}_{ik}}{{\sum }_{{k}^{t} = 1}^{n} {l}_{{ik}^{′}}} {bf}_{kji} & ( 2 )\end{matrix}$$

Here, BFⱼᵢ on the left side indicates a j^{th} body-movement feature of a measurement subject i, and the right side for the measurement subject i indicates that a weighted average of features bfₖⱼᵢ (the numerical value of the j^{th} feature of the measurement subject i in a k^{th} measurement among a total of n measurements) calculated in the respective measurements is calculated using a length lᵢₖ (6 hours, 7 hours, and 8 hours in the above-described example) of the k^{th} measurement.

The going-out frequency calculation unit 104 calculates an estimated value of a going-out frequency of the measurement subject based on activity data of the measurement subject inferred from the body-movement data stored in the body-movement data storage unit 102. The activity data is data indicating the ratio of a specific activity among all activities of each individual measurement subject. The activity data is calculated by: obtaining, for each individual measurement subject, a histogram indicating the frequency of each activity state based on a moving average obtained from a time-series change in the body-movement data; further calculating thresholds for distinguishing between the activity states using the obtained histogram; and using the calculated thresholds. Alternatively, the activity data is data indicating the ratio of a specific activity when the moving average obtained from the time-series change in the body-movement data is greater than or equal to a threshold that is common among the measurement subjects. For example, for a measurement subject who is an office worker working at his/her desk, the ratio of walking or running (specific activity) in the office is low, and it can be generally assumed that the measurement subject has gone out of the office if walking or running is observed. Thus, the going-out frequency corresponds to the temporal ratio (activity data) of the walking or running state estimated from the body-movement data, or the like. However, the going-out frequency is not limited to this. When defining the going-out frequency as the total of the ratios of the walking state and the running state, the temporal ratio of times when an activity magnitude indicated by RMS_{ACC} (specifically, the left side of formula (4)) indicating body-movement intensity obtained by calculation formulae such as those in formulae (3) and (4) below is greater than or equal to a threshold that is the same for all users can be set as the going-out frequency, as in Non-Patent Document 1 for example. Alternatively, as disclosed in Non-Patent Document 2, walking and running may be distinguished from one another using a threshold individually derived from an RMS_{ACC} histogram for an individual user.

$$\begin{matrix}[ Math . 3 ] & \\ {\overline{a}}_{{x}_{i} {t}_{1}} = {a}_{{x}_{i} {t}_{1}} - \frac{1}{{T}_{1}} {\sum }_{{t}_{0} = {t}_{1} - {T}_{1}}^{{t}_{1}} {a}_{{x}_{i} {t}_{0}} , ( i = 1 , 2 , 3 ) & ( 3 )\end{matrix}$$

$$\begin{matrix}[ Math . 4 ] & \\ \overline{{RMS}_{ACC}} ( {t}_{2} ) = \frac{1}{{T}_{2}} {\sum }_{{t}_{1} = {t}_{2} - {T}_{2}}^{{t}_{2}} \sqrt{{( {\overline{a}}_{{x}_{1} {t}_{1}} )}^{2} + {( {\overline{a}}_{{x}_{2} {t}_{1}} )}^{2} + {( {\overline{a}}_{{x}_{3} {t}_{1}} )}^{2}} & ( 4 )\end{matrix}$$

In formula (3), x₁, x₂, and x₃ indicate three axes (x, y, and z axes, etc.) in a space, and a to which x₁, x₂, and x₃ are appended as subscripts indicates acceleration signals in the directions along the three axes. Furthermore, t₀ further appended as a subscript to a indicates the time when the acceleration signals were acquired by the wearable terminal 200. Formula (3) indicates, in regard to each of the three axes, the degree of change in the acceleration signal at time t₁ as compared to the moving average of the acceleration signal from time t₁-T₁ to time t₁.

Furthermore, in formula (4), the moving average, at time t₂, of the rooted mean square (RMS) of the individual degrees of change in the three axes from time t₂-T₂ to time t₂ is calculated. As a whole, by including moving averages in the stage of individual calculations for the RMS of the degrees of change of the acceleration signals, an average value of the change in acceleration over a certain time period can be calculated instead of a spontaneous change in acceleration, whereby activity states can be determined accurately.

Non-Patent Document 2 also discloses the approach of distinguishing body-movement features themselves from one another using RMS_{ACC}. According to this approach, instead of indicating the entire data acquisition period, lᵢₖ indicated in formula (2) indicates the specific sitting, walking, and running times included in the data acquisition period. For example, when using the same example as that in the explanation of formula (2) (i.e., the example in which data was acquired over a 3-day period) and supposing that during the 3-day data acquisition period: a 0.5-hour running period was included in the six hours of day 1 and the average value of body movement during the period was 1.6 G; a 0.4-hour running period was included in day 2 and the average value of body movement during the period was 1.8 G; and a 0.2-hour running period was included in day 3 and the average value of body movement during the period was 2.2 G, the average value in such a case should be 1.6*0.5/(0.5+0.4+0.2)+1.8*0.4/(0.5+0.4+0.2)+2.2*0.2/(0.5+0.4+0.2). A feature based on this approach is indicated in mathematical formula (5) below.

$$\begin{matrix}[ Math . 5 ] & \\ {BF}_{ji} ( run ) = {\sum }_{k = 1}^{n} \frac{{l}_{ik} ( run )}{{\sum }_{{k}^{′} = 1}^{n} {l}_{{ik}^{′}} ( run )} {bf}_{kji} ( run ) & ( 5 )\end{matrix}$$

The above corresponds to running. However, the feature for walking and the feature for sitting can also be defined similarly (see mathematical formulae (6) and (7) below).

$$\begin{matrix}[ Math . 6 ] & \\ {BF}_{ji} ( walk ) = {\sum }_{k = 1}^{n} \frac{{l}_{ik} ( walk )}{{\sum }_{{k}^{′} = 1}^{n} {l}_{{ik}^{′}} ( walk )} {bf}_{kji} ( walk ) & ( 6 )\end{matrix}$$

$$\begin{matrix}[ Math . 7 ] & \\ {BF}_{ji} ( sit ) = {\sum }_{k = 1}^{n} \frac{{l}_{ik} ( sit )}{{\sum }_{{k}^{′} = 1}^{n} {l}_{{ik}^{′}} ( sit )} {bf}_{kji} ( sit ) & ( 7 )\end{matrix}$$

Based on such an approach, when defining GFᵢ as the going-out frequency of an individual i among an N number of measurement subjects, GFᵢ can be defined as indicated in formula (8) below based on formulae (4) and (5).

$$\begin{matrix}[ Math . 8 ] & \\ {GF}_{i} = {\sum }_{k = 1}^{n} {l}_{ik} ( walk ) + {\sum }_{k = 1}^{n} {l}_{ik} ( sun ) & ( 8 )\end{matrix}$$

Formula (8) is merely one example, and data acquired from a source other than the wearable terminal 200, such as the measurement subject's schedule data, office entry/exit record data, or transportation use record data, may be used, as long as the amount reflects the frequency at which an office worker goes out of the office. Furthermore, the going-out frequency may be based on a client visit report, etc., submitted by the measurement subject himself/herself.

The body-movement feature correction unit 105 corrects the correlation of the body-movement feature with stress using the body-movement feature and the going-out frequency. As the method of the correction by the body-movement feature correction unit 105, the correction can be performed by, as shown in formula (9), obtaining BFⱼᵢ′ for a numerical value BFⱼᵢ of a j^{th} body-movement feature in i by using the going-out frequency GFᵢ defined in formula (8).

$$\begin{matrix}[ Math . 9 ] & \\ {BF}_{ji}^{′} = {{BF}_{ji} ( \frac{{GF}_{i}}{\frac{1}{N} {\sum }_{{i}^{′} = 1}^{N} {GF}_{{i}^{′}}} )}^{a} & ( 9 )\end{matrix}$$

Here, a is a negative value such as −1. The correction based on formula (9) is merely one example. As long as the mathematical formula to be used is that with which the body-movement feature can be corrected so as to become greater for measurement subjects having a low going-out frequency, the scope of the present application not only includes setting a to −1 and simply dividing the body-movement feature by a correction term (the formula to the right of BFⱼᵢ in formula (9)) but also includes setting α to an arbitrarily defined negative value other than −1, and calculation operations other than that in formula (9).

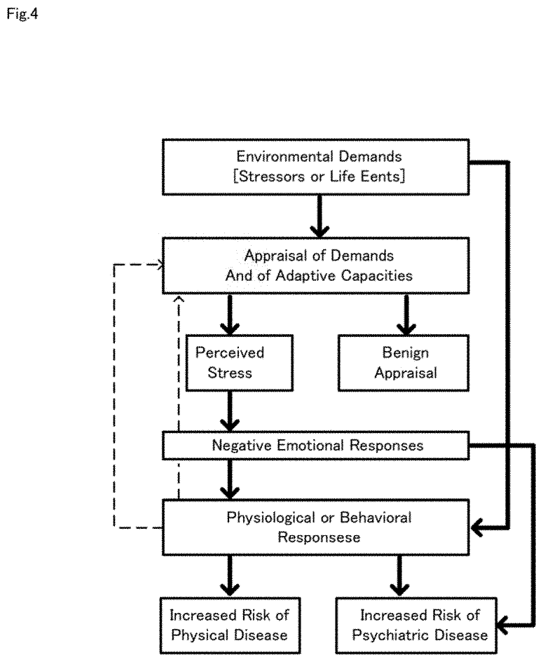

The reason why the stress feature can be corrected using a calculation operation, such as that in formula (9), with which the body-movement feature can be corrected so as to become greater for measurement subjects having a low going-out frequency will be described in further detail with reference to FIG. 4.

FIG. 4 is a diagram for explaining why the stress feature can be corrected.

In FIG. 4, the items indicated as environmental demands are stressors. For office workers, stressors differ depending on job type, etc., because there are both a case in which frequent visits to clients are the main stressor and a case in which the difficulty of work that the office worker is working on in the office, etc., is the main stressor. The “going-out frequency” in the present application is used to derive, from actual data, differences in environmental demands that are attributable to such differences in job type, etc.

For example, in formula (1), a situation in which the body-movement feature is small relative to the stress score (the “degree of contribution” of the body-movement feature is small) indicates a situation in which environmental demands are not related to the action of going out. This situation is typical for a measurement subject having a low going-out frequency and high stress, for example. However, as in formula (8), the body-movement feature of such a subject can be made higher by correcting the body-movement feature of the measurement subject by multiplying the body-movement feature by the reciprocal of the relative going-out frequency (going-out frequency compared with the average of that of other measurement subjects), etc. For such a measurement subject, the difficulty of the work that the measurement subject is working on in the office, rather than the action of visiting clients, is the actual stressor, and environmental demands are high due to this. Nevertheless, correction is virtually performed as through the action of going out (the action of visiting clients) is an environmental demand. By performing such an operation, a model can be formed regarding that the measurement subjects, as a whole, form a group having an attribute that the action of going out (the action of visiting clients) is the main environmental demand.

As explained with reference to formulae (5), (6), and (7), all body-movement features are normalized by the durations of the activity states (sitting, walking, and running). Thus, there is no direct relation between the temporal lengths of activity states (going-out frequency) and the magnitude of the body-movement feature. The body-movement feature simply reflects the intensity of body movement in the individual activity states.

If the action of visiting clients or the like is the main environmental demand, it can be considered that the magnitude of the body-movement feature at the site of visit (e.g., in a case in which an acceleration signal is adopted as the body-movement signal, the high ratio of great acceleration in a time-series histogram of acceleration; i.e., the high ratio of running and walking in a hurry) has a higher correlation with stress than the frequency (temporal length) of visits to clients.

In contrast, if work in the office while sitting is the main stressor, the body-movement feature would be substantially unrelated with stress. On the other hand, it can be considered that the duration of work performed while sitting (the low ratio of walking and running) is related with a stressor.

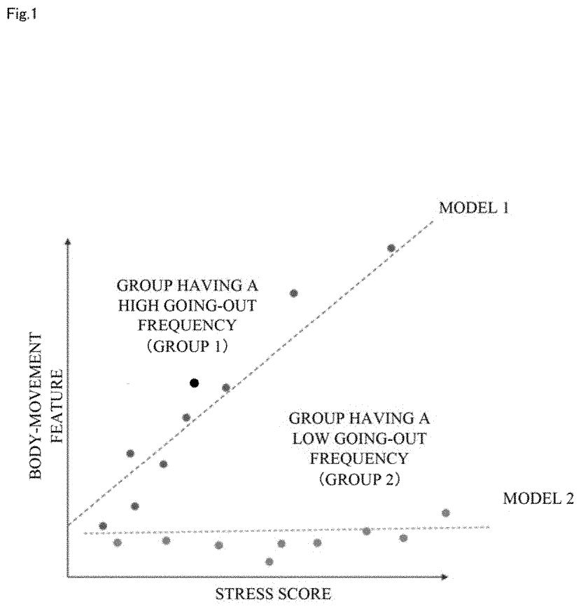

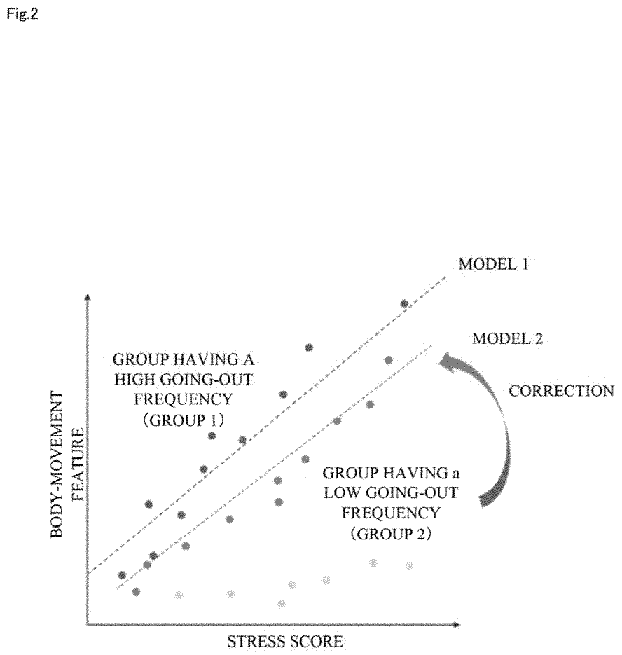

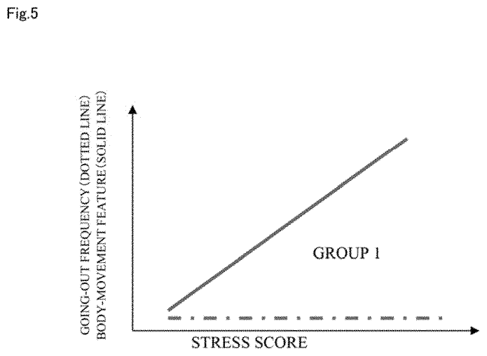

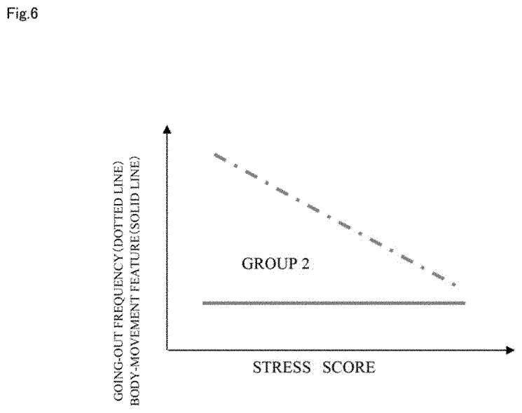

When such circumstances are summarized, it can be considered that the relation between environmental demands and the going-out frequency would be as illustrated in FIGS. 5 and 6. FIGS. 5 and 6 are diagrams illustrating how stress is related with the going-out frequency and the body-movement feature. Group 1 in FIG. 5 and group 2 in FIG. 6 are the same as groups 1 and 2 in FIGS. 1 and 2. As illustrated in FIG. 5, in group 1, the going-out frequency is constant, but the body-movement feature increases proportionately as stress increases. In contrast, in group 2, while the going-out frequency decreases proportionately as stress increases, the body-movement feature is constant as illustrated in FIG. 6.

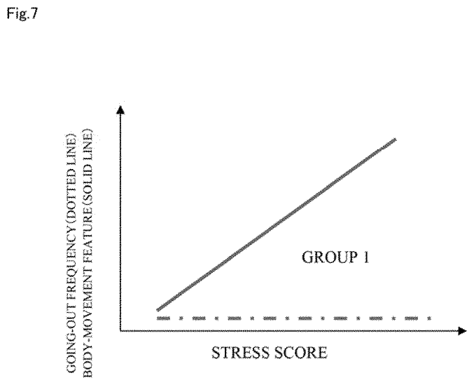

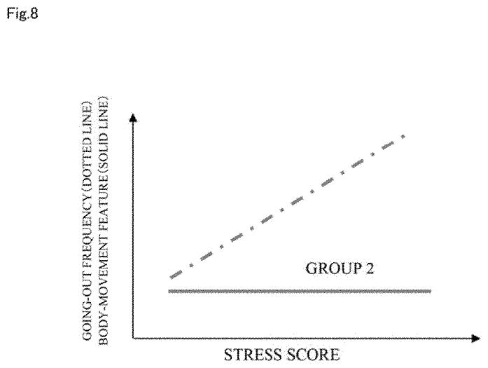

FIG. 7 is a diagram in which, in place of the going-out frequency in FIG. 5, the reciprocal of the going-out frequency is illustrated. FIG. 8 is a diagram in which, in place of the going-out frequency in FIG. 6, the reciprocal of the going-out frequency is illustrated.

As illustrated in FIG. 7, in group 1, the body-movement feature is proportional to stress, and the going-out frequency is substantially constant. Furthermore, as illustrated in FIG. 8, in group 2, the going-out frequency is proportional to stress, and the body-movement feature is substantially constant. By multiplying these indicators, a feature proportional to stress can be obtained for both group 1 and group 2. In practice, information indicating whether each individual measurement subject belongs to group 1 or group 2 cannot be obtained in advance, and thus an operation in which the body-movement feature is used for group 1 and the reciprocal of the going-out frequency is used for group 2 cannot be performed. Thus, it is suitable to use an indicator obtained by multiplying these two indicators. However, provided that such information can be obtained in advance, an operation such as that in which the body-movement feature and the reciprocal of the going-out frequency are used as the stress estimation features for group 1 and group 2, respectively, is also included within the scope of the present application.

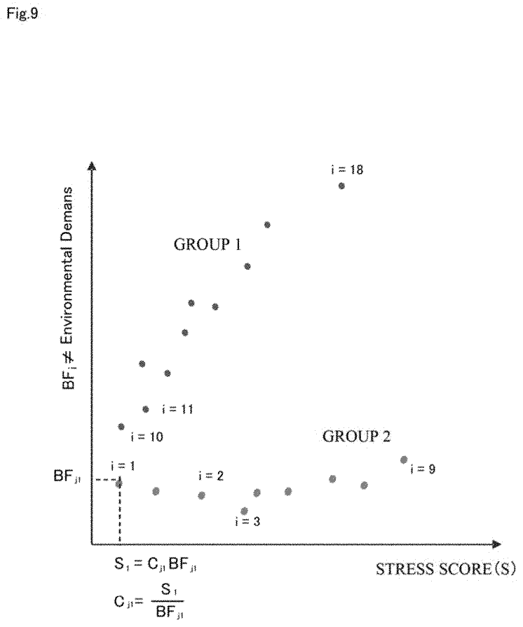

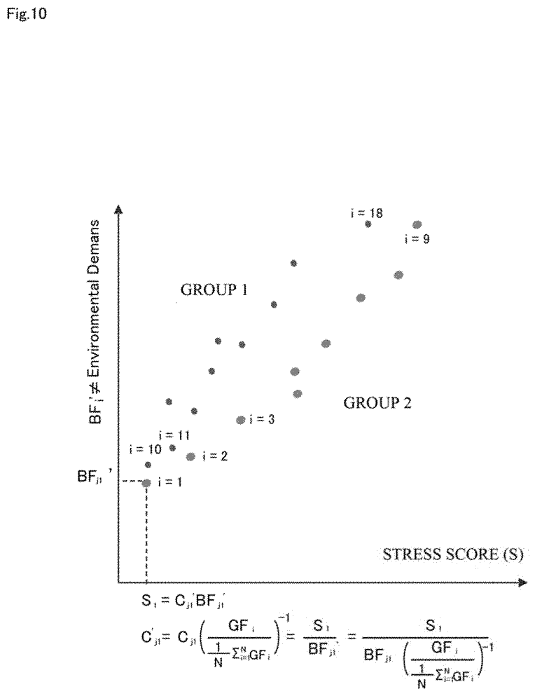

Reference will be made to FIGS. 9 and 10 to explain the operation in formula (8) in further detail. FIGS. 9 and 10 are diagrams for explaining the operation in formula (8) in further detail.

FIG. 9 illustrates plots of the body-movement feature BFⱼᵢ and the stress score Sᵢ before the correction in formula (8) is performed. As illustrated in the schematic diagram in FIG. 4, it can be considered that environmental demands and the stress score Sᵢ are proportional to one another. Thus, the “degree of contribution” is constant. However, in the group having a low going-out frequency (group 2), the “degree of contribution” Cⱼᵢ of BFⱼᵢ to stress scores is not constant, and it can be considered that the correlation of BFⱼᵢ with stress scores is low. The “degree of contribution” Cⱼᵢ in group 1 decreases as stress increases as in the explanation provided in FIGS. 5 to 8, whereas the going-out frequency decreases as stress increases.

Here, as illustrated in FIG. 10, for group 2, a corrected body-movement feature BFⱼᵢ′ is obtained by multiplying, with the body-movement feature BFⱼᵢ, a correction term in which a is set to −1 in formula (8). By doing so, a corrected degree of contribution Cⱼᵢ′ corresponding to the corrected body-movement feature becomes substantially constant. While the same operation is performed for group 1 as well, not much change occurs even if correction is performed using the reciprocal of the going-out frequency since the going-out frequency is constantly high.

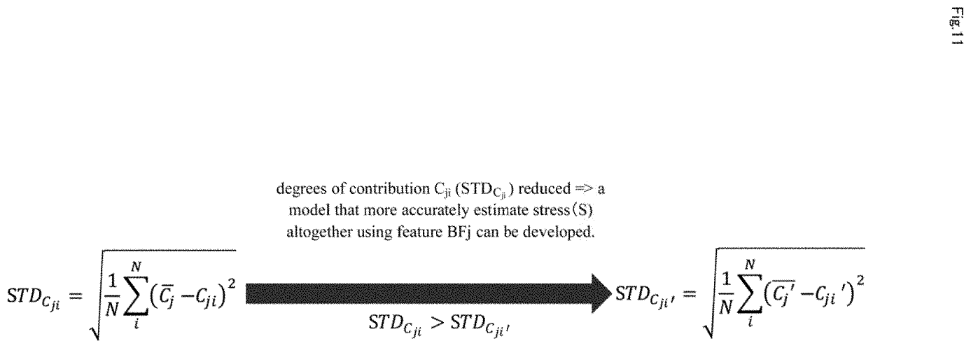

FIG. 11 is a diagram illustrating the operation performed to improve the accuracy of the model for estimating stress scores. As illustrated in FIG. 11, by performing such an operation, the variance (standard deviation) of Cⱼᵢ that was great in FIG. 9 becomes relatively small in FIG. 10. Furthermore, this variance (standard deviation) is calculated to include all of the “degrees of contribution” Cⱼᵢ in group 1 and group 2. Thus, by this variance (standard deviation) being reduced by performing such an operation, a model that more accurately estimate stress scores for group 1 and group 2 altogether using the body-movement feature BFⱼᵢ′ can be developed.

The corrected body-movement feature output unit 106 outputs the body-movement feature corrected by the body-movement feature correction unit 105.

The stress estimation unit 107 estimates stress using the corrected body-movement feature. The stress estimation unit 107 estimates stress using only the body-movement feature output from the corrected body-movement feature output unit 106, or estimates stress also using a stress feature calculated from a biosignal other than body movement in addition to the body-movement feature.

[Apparatus Operations]

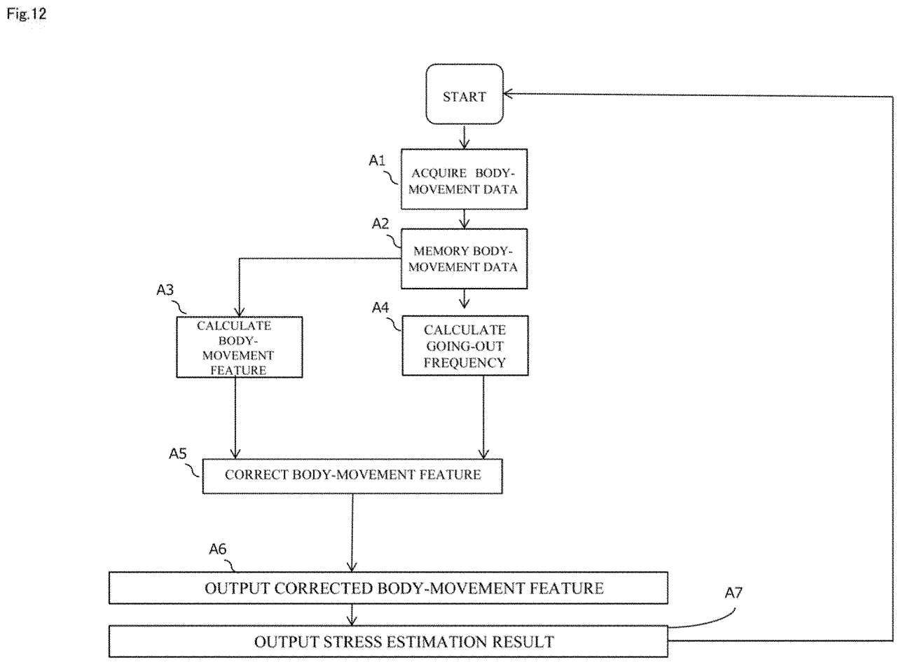

Next, operations of the stress estimation apparatus 100 in the present example embodiment will be described with reference to FIG. 12. FIG. 12 is a flowchart illustrating operations of the stress estimation apparatus 100. In the present example embodiment, a stress estimation method is implemented by causing the stress estimation apparatus 100 to operate. Accordingly, the following description of the operations of the stress estimation apparatus 100 is substituted for the description of the stress estimation method in the present example embodiment.

The body-movement data acquisition unit 101 acquires body-movement data transmitted from the wearable terminal 200 (step A1), and stores the acquired body-movement data to the body-movement data storage unit 102 (step A2). The body-movement feature calculation unit 103 calculates a body-movement feature from the body-movement data (step A3). The going-out frequency calculation unit 104 calculates a going-out frequency based on formula (8), etc. (step A4). The body-movement feature correction unit 105 corrects the going-out frequency based on formula (9) (step A5). The corrected body-movement feature output unit 106 outputs the corrected feature (step A6). The stress estimation unit 107 estimates stress using this corrected body-movement feature (step A6).

[Program]

It suffices for a program in the present example embodiment to be a program that causes a computer to carry out steps A1 to A7 illustrated in FIG. 12. By installing this program on a computer and executing the program, the stress estimation apparatus 100 and the stress estimation method in the present example embodiment can be realized. In this case, a processor of the computer functions and performs processing as the body-movement data acquisition unit 101, the body-movement data storage unit 102, the body-movement feature calculation unit 103, the going-out frequency calculation unit 104, the body-movement feature correction unit 105, the corrected body-movement feature output unit 106, and the stress estimation unit 107.

[Effects of Embodiment]

In the present example embodiments described above, stress can be estimated using the same model regardless of the going-out frequency of a measurement subject.

### EXAMPLE

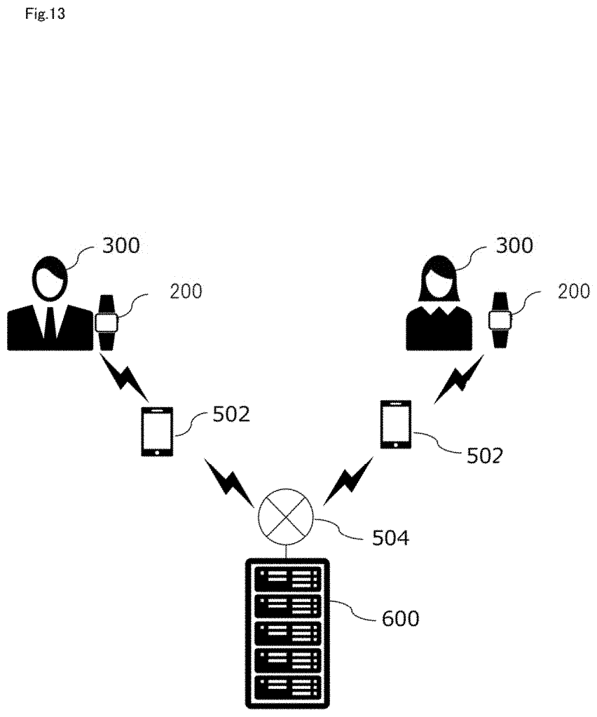

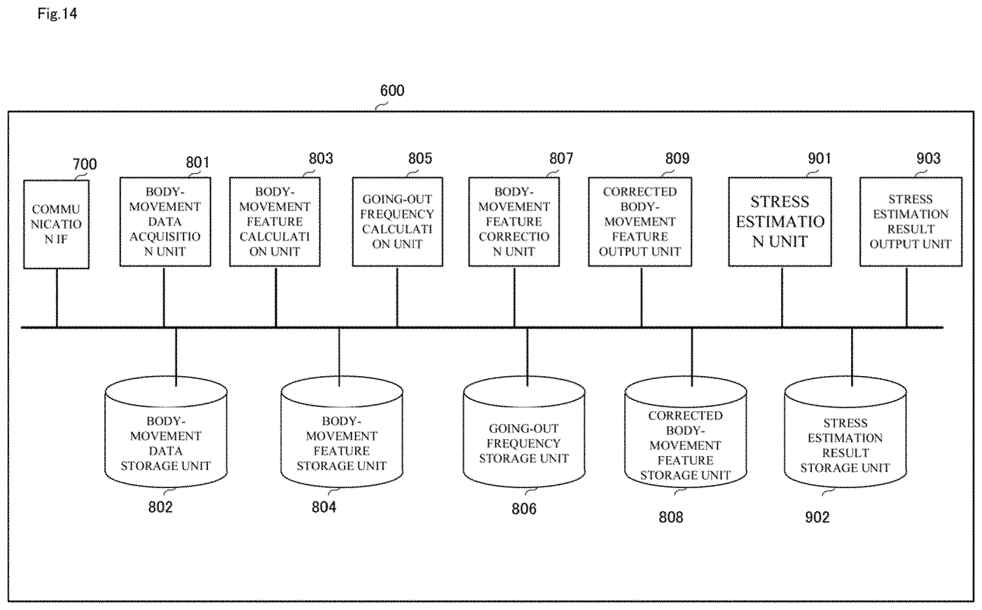

An example of the present example embodiment will be described in detail with reference to FIGS. 13 and 14. FIGS. 13 and 14 are diagrams for describing a specific example of the present example embodiment. In this example, description is provided regarding that the stress estimation apparatus 100 is a computer 600 connected to the Internet 504.

As illustrated in FIG. 13, the computer 600 is configured so as to communicate with the wearable terminal 200 worn by each measurement subject 300 via a portable terminal 502 owned by the measurement subject 300. The portable terminal 502 and the wearable terminal 200 perform the transmission and reception of data with one another via Bluetooth (registered trademark), for example. Furthermore, the portable terminal 502 and the computer 600 perform the transmission and reception of data with one another via packet communication, for example.

The wearable terminal 200 acquires a biosignal reflecting bio-information of a measurement subject 300, in addition to acceleration in three axes reflecting body movement of the measurement subject 300. As the biosignal of the measurement subject 300, all types of bio-information affected by the mental activities of the measurement subject are included within the scope of the invention. Bio-information of the measurement subject 300 includes, besides electrodermal activity, which reflects sweating of the measurement subject 300 as mentioned in Non-Patent Document 1, body temperature, pulse wave, heart rate, voice, brain waves, respiration, myoelectricity, and cardioelectricity, and also acceleration signals reflecting body movement, etc.

As mentioned above, the wearable terminal 200 itself may be any wearable device as long as the body-movement signal and one of the biosignals reflecting the types of bio-information mentioned above can be measured, and the wearable terminal 200 can be worn by the measurement subject, such wearable devices including, besides a wrist-band-type wearable device such as that disclosed in Non-Patent Document 1, a badge-type wearable device, an ID-card-type wearable device, an earphone-type wearable device, a shirt-type wearable device, a head-mounted-type wearable device, an eyeglass-type wearable device, etc. Specifically, in this working example, the wearable terminal acquires only the acceleration signals, which are one type of a body-movement signal, at a predetermined sampling rate, and stores the acceleration signals to an internal memory.

The wearable terminal 200 transmits the acceleration signal data and the biosignal data that have been acquired to the computer 600 via the portable terminal 502. Specifically, the wearable terminal 200 transmits the biosignal data to the portable terminal 502 by connecting to the portable terminal 502 via Bluetooth (registered trademark). Subsequently, the biosignal data is transmitted via packet communication to the Internet 504 by an application installed on the portable terminal 502, and is uploaded to the computer 600 on the Internet 504.

As illustrated in FIG. 14, a communication interface 700, data processing elements, and data storage elements are present in the computer 600. A body-movement data acquisition unit 801, a body-movement feature calculation unit 803, a going-out frequency calculation unit 805, a body-movement feature correction unit 807, a corrected body-movement feature output unit 809, a stress estimation unit 901, and a stress estimation result output unit 903 are present as data processing elements. Furthermore, a body-movement data storage unit 802, a body-movement feature storage unit 804, a going-out frequency storage unit 806, a corrected body-movement feature storage unit 808, and a stress estimation result storage unit 902 are present as data storage elements.

First, the body-movement data obtained from the communication interface 700 is stored to the body-movement data storage unit 802 via the body-movement data acquisition unit 801. Next, the body-movement feature calculation unit 803 calculates a body-movement feature using the body-movement data obtained from the body-movement data storage unit 802. This data is stored to the body-movement feature storage unit 804. Next, the going-out frequency calculation unit 805 calculates a going-out frequency using the temporal ratio of walking and running, etc. The calculated going-out frequency is stored to the going-out frequency storage unit 806.

Next, the body-movement feature correction unit 807 corrects the body-movement feature based on a calculation formula such as formula (9) by using the body-movement feature and the going-out frequency stored in the body-movement feature storage unit 804 and the going-out frequency storage unit 806. Thus, the body-movement feature becomes a numerical value that has a stronger correlation with the stress score.

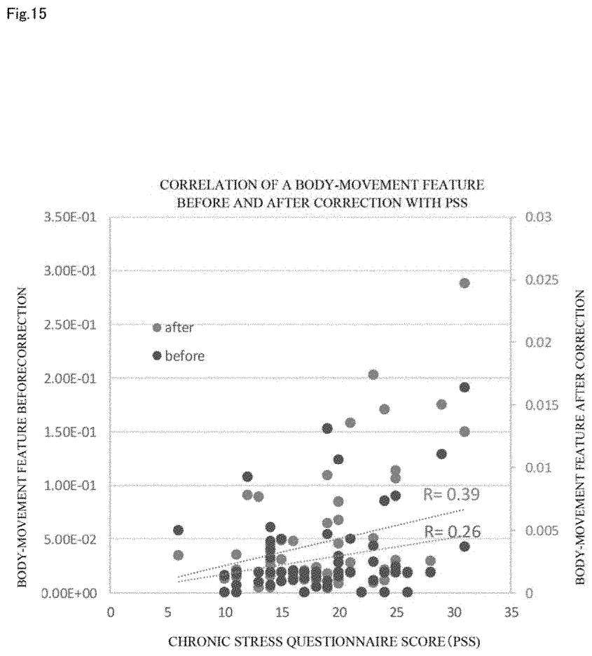

Specifically, description will be provided with reference to FIG. 15, which illustrates results obtained through analysis of data obtained by carrying out an experiment according to the first working example. FIG. 15 is a diagram illustrating the correlation of a body-movement feature before and after correction with PSS. FIG. 15 illustrates a comparison of numerical values of chronic stress scores called Perceived Stress Scale (PSS) scores before and after one body-movement feature (time-series histogram feature during running) is corrected. As illustrated in FIG. 15, a great improvement is observed, with the correlation coefficient after correction being 0.39 while the correlation coefficient before correction is 0.26.

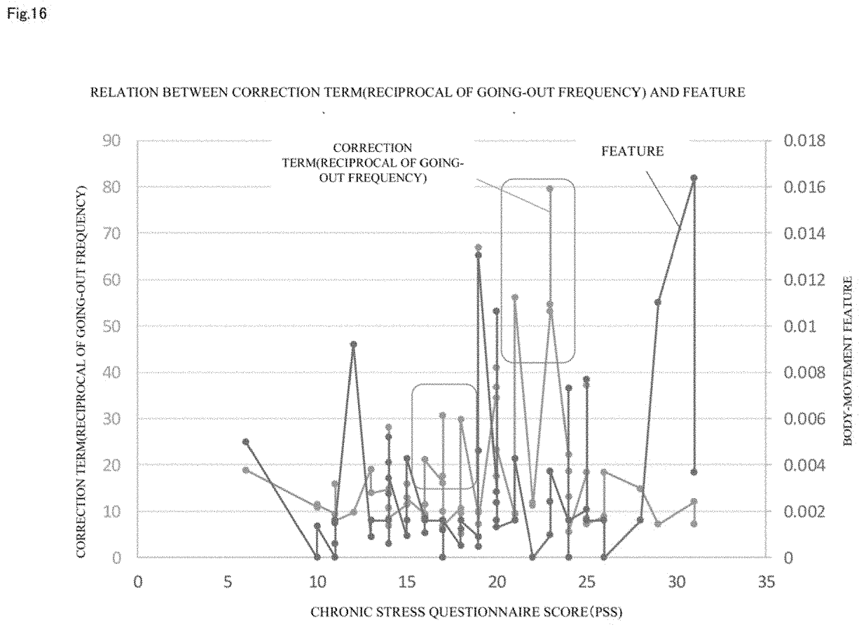

FIG. 16 is a diagram illustrating the relation between the correction term and the body-movement feature. The reason for the above can be explained from the situation (the framed portion in FIG. 16) in which the correction term (reciprocal of the going-out frequency) is complementarily great (i.e., the going-out frequency is low) for a person having a low body-movement feature numerical value despite having a high chronic stress questionnaire score, as illustrated in FIG. 16. A person for which the correction term (reciprocal of the going-out frequency) is great is a person corresponding to group 2 who does not go out much, and the correlation of the body-movement feature with the stress score is low. No further processing needs to be performed if a stress estimation model is to be created for only such people. However, as described above, in order to perform analysis using the same feature and model as people corresponding to group 1 who have a high going-out frequency and for which the correlation of the body-movement feature with the stress score is high, an operation for artificially increasing the correlation of the body-movement feature of people having a low going-out frequency is necessary. Conversely, in order to analyze people having a high going-out frequency using the same model as that for people having a low going-out frequency, an operation for artificially decreasing the correlation of the body-movement feature of such people with stress is necessary. FIG. 16 explains such a situation, and the effect of the operation is shown by FIG. 15.

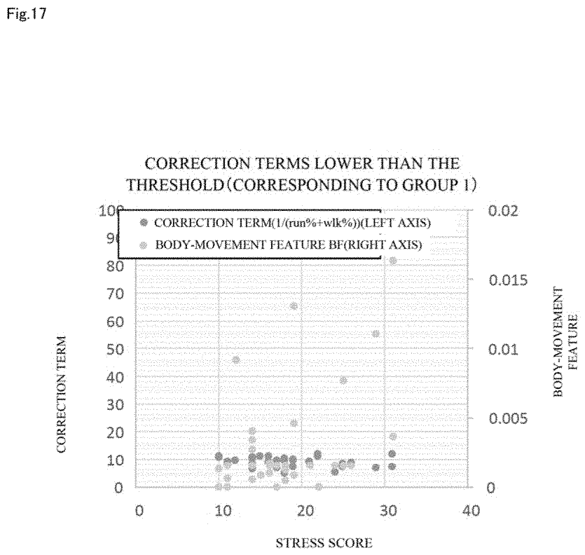

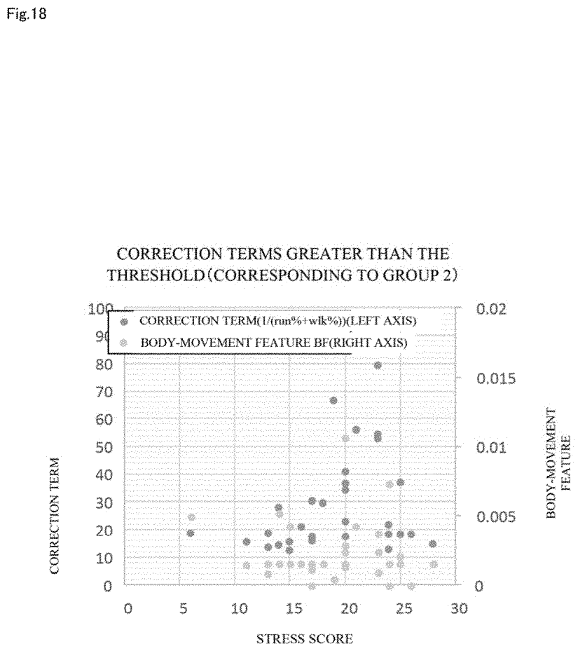

FIG. 17 is a graph in which the schematic graph illustrated in FIG. 7 is verified with real data. FIG. 18 is a graph in which the schematic graph illustrated in FIG. 8 is verified with real data.

FIG. 17 is a diagram in which 12, which is a numerical value obtained by rounding off the median of correction terms in data for a total of 64 people illustrated in FIG. 16, is used as a threshold, and data having correction terms lower than the threshold is illustrated as data corresponding to group 1. FIG. 18 is a diagram in which 12, which is a numerical value obtained by rounding off the median of correction terms in data for a total of 64 people illustrated in FIG. 16, is used as a threshold, and data having correction terms lower than the threshold is illustrated as data corresponding to group 2.

In FIG. 17, while the correction term has substantially constant numerical values, a trend can be observed in which the body-movement feature is proportional to stress scores. On the other hand, in FIG. 18, while the correction term exhibits a trend of being proportional to stress scores, the body-movement feature exhibits a trend of remaining at constant numerical values although there is a slight variation.

Returning to FIG. 14, the corrected feature is stored to the corrected body-movement feature storage unit 808.

Next, the corrected body-movement feature is output from the corrected body-movement feature output unit 809 to the stress estimation unit 901.

The stress estimation unit 901 estimates stress, and stores the estimation result to the stress estimation result storage unit 902. The stress estimation by the stress estimation unit 901 can be realized by creating a model for estimating PSS scores by regression analysis using PSS scores as correct values of stress, for example. In doing so, a machine learning model such as an SVM model is trained using scores calculated from a PSS questionnaire given to measurement subjects at the end of an experiment period (four weeks) as training data, and using the corrected body-movement feature as the stress feature. A PSS score can be estimated using a model created in such a manner, and the PSS score can be set as a stress estimation result.

Next, in response to a request by the measurement subject, the stress estimation result output unit 903 outputs the stress estimation result in the stress estimation result storage unit 902. For example, outputting on a screen, outputting by printing, etc., can be mentioned as examples of the method in which the outputting is performed, but there is no limitation to such methods. As examples of the output timing, outputting can be performed at all times or in response to requests by the measurement subject. Specifically, when outputting on a screen is to be performed, the stress estimation result stored in the stress estimation result storage unit 902 is transmitted to the wearable terminal 200 or the portable terminal 502 via the communication interface 700, and is output from a screen attached to the wearable terminal 200 or portable terminal 502.

### (Physical Configuration of Apparatus)

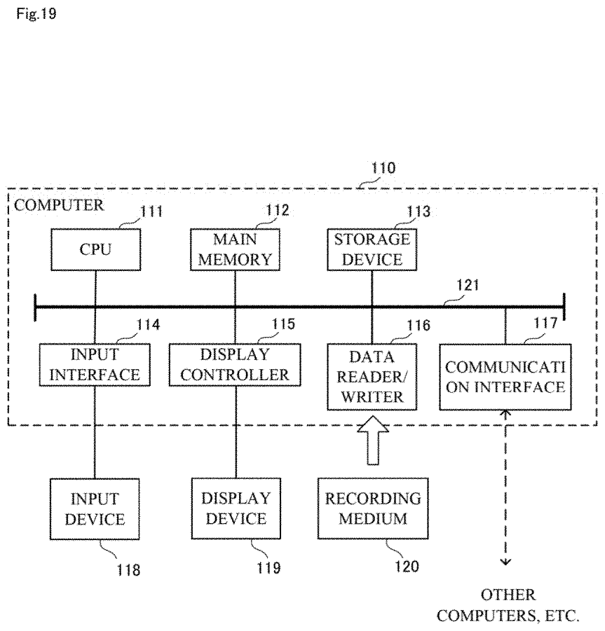

Using FIG. 19, a description is now given of a computer that realizes the stress estimation apparatus 100 by executing the program in the example embodiment will be described. FIG. 19 is a block diagram showing one example of a computer that realizes the stress estimation apparatus 100 in the present example embodiment.

As shown in FIG. 19, a computer 110 includes a CPU 111, a main memory 112, a storage device 113, an input interface 114, a display controller 115, a data reader/writer 116, and a communication interface 117. These components are connected in such a manner that they can perform data communication with one another via a bus 125. Note that the computer 110 may include a GPU (Graphics Processing Unit) or an FPGA (Field-Programmable Gate Array) in addition to the CPU 111 or in place of the CPU 111.

The CPU 111 carries out various types of computation by deploying the program (codes) in the present example embodiment stored in the storage device 113 to the main memory 112, and executing the deployed program in a predetermined order. The main memory 112 is typically a volatile storage device, such as a DRAM (Dynamic Random Access Memory). Also, the program in the present example embodiment is provided in a state where it is stored in a computer readable recording medium 120. Note that the program in the present example embodiment may also be distributed over the Internet connected via the communication interface 117.

Furthermore, specific examples of the storage device 113 include a hard disk drive, and also a semiconductor storage device, such as a flash memory. The input interface 114 mediates data transmission between the CPU 111 and an input apparatus 118, such as a keyboard and a mouse. The display controller 115 is connected to a display apparatus 119, and controls displays on the display apparatus 119. The data reader/writer 116 mediates data transmission between the CPU 111 and the recording medium 120, and executes readout of the program from the recording medium 120, as well as writing of the result of processing in the computer 110 to the recording medium 120. The communication interface 117 mediates data transmission between the CPU 111 and other computers.

Also, specific examples of the recording medium 120 include: a general-purpose semiconductor storage device, such as CF (Compact Flash®) and SD (Secure Digital); a magnetic recording medium, such as Flexible Disk; and an optical recording medium, such as CD-ROM (Compact Disk Read Only Memory).

A part or all of the aforementioned example embodiment can be described as, but is not limited to, the following (Supplementary Note 1) to (Supplementary Note 24).

(Supplementary Note 1)

A stress estimation apparatus that estimates stress of a measurement subject, comprising:

a body-movement data acquisition unit for acquiring body-movement data;

a body-movement data storage unit for storing body-movement data;

a body-movement feature calculation unit for calculating a stress-related body-movement feature from the stored body-movement data;

a going-out frequency calculation unit for calculating an estimated value of a going-out frequency from the stored body-movement data;

a body-movement feature correction unit for correcting a correlation of the body-movement feature with stress using the body-movement feature and the going-out frequency;

a corrected body-movement feature output unit for outputting the corrected body-movement feature; and

a stress estimation unit for estimating stress using the corrected body-movement feature.

(Supplementary Note 2)

The stress estimation apparatus according to Supplementary note 1, wherein

the going-out frequency calculation unit

calculates the estimated value of the going-out frequency based on activity data of the measurement subject inferred from the body-movement data.

(Supplementary Note 3)

The stress estimation apparatus according to Supplementary note 2, wherein

the activity data is data indicating the ratio of a specific activity among all activities performed by each individual measurement subject, and

the activity data is calculated by:

obtaining, for each individual measurement subject, a histogram indicating the frequency of each activity state based on a moving average obtained from a time-series change in the body-movement data;

further calculating a threshold for distinguishing each activity state using the obtained histogram; and

using the calculated threshold.

(Supplementary Note 4)

The stress estimation apparatus according to Supplementary note 2, wherein

the activity data is data indicating the ratio of a specific activity when a moving average obtained from a time-series change in the body-movement data is greater than or equal to a threshold that is common among measurement subjects.

(Supplementary Note 5)

The stress estimation apparatus according to Supplementary note 1, wherein

the going-out frequency calculation unit

calculates the estimated value of the going-out frequency based on the measurement subject's schedule data, office entry/exit record data, or transportation use record data, in place of the body-movement data.

(Supplementary Note 6)

The stress estimation apparatus according to Supplementary note 1, wherein

the going-out frequency calculation unit

calculates the estimated value of the going-out frequency based on a report by the measurement subject himself/herself, in place of the body-movement data.

(Supplementary Note 7)

The stress estimation apparatus according to any one of Supplementary notes 1 to 6, wherein

the going-out frequency calculation unit calculates the estimated value of the going-out frequency for a plurality of measurement subjects, and

the body-movement feature correction unit corrects the correlation of the body-movement feature with stress by performing multiplication with the reciprocal of a ratio of an average value for the measurement subject to an average value of the estimated values of the going-out frequencies of all measurement subjects.

(Supplementary Note 8)

The stress estimation apparatus according to any one of Supplementary notes 1 to 7, wherein

the stress estimation unit estimates stress using a feature calculated from a signal other than the body-movement data.

(Supplementary Note 9)

A stress estimation method for estimating stress of a measurement subject, comprising:

a step of acquiring body-movement data;

a step of storing body-movement data;

a step of calculating a stress-related body-movement feature from the stored body-movement data;

a step of calculating an estimated value of a going-out frequency from the stored body-movement data;

a step of correcting a correlation of the body-movement feature with stress using the body-movement feature and the going-out frequency;

a step of outputting the corrected body-movement feature; and

a step of estimating stress using the corrected body-movement feature.

(Supplementary Note 10)

The stress estimation method according to Supplementary note 9, wherein

in the step of calculating the estimated value of the going-out frequency,

the estimated value of the going-out frequency is calculated based on activity data of the measurement subject inferred from the body-movement data.

(Supplementary Note 11)

The stress estimation method according to Supplementary note 10, wherein

the activity data involves calculating a threshold for each individual measurement subject, wherein the calculation method of the threshold includes forming a histogram from numerical value data of a moving average of the change in body-movement data within a predetermined period and calculating a threshold for each activity state for each individual using the histogram, and a ratio of a specific activity is adopted as the activity data.

(Supplementary Note 12)

The stress estimation method according to Supplementary note 10, wherein

the activity data involves calculating a threshold that is common among measurement subjects, and the calculation method involves making a determination based on whether or not a moving average of the change in the body-movement data has exceeded a given threshold.

(Supplementary Note 13)

The stress estimation method according to Supplementary note 9, wherein

in the step of calculating the estimated value of the going-out frequency,

the estimated value of the going-out frequency is calculated based on the measurement subject's schedule data, office entry/exit record data, or transportation use record data, in place of the body-movement data.

(Supplementary Note 14)

The stress estimation method according to Supplementary note 9, wherein

in the step of calculating the estimated value of the going-out frequency,

the estimated value of the going-out frequency is calculated based on a report by the measurement subject himself/herself, in place of the body-movement data.

(Supplementary Note 15)

The stress estimation method according to any one of Supplementary notes 9 to 14, wherein

in the step of calculating the estimated value of the going-out frequency, the estimated value of the going-out frequency is calculated for a plurality of measurement subjects, and

in the step of correcting the correlation, the correlation of the body-movement feature with stress is corrected by performing multiplication with the reciprocal of a ratio of an average value for the measurement subject to an average value of the estimated values of the going-out frequencies of all measurement subjects.

(Supplementary Note 16)

The stress estimation method according to any one of Supplementary notes 9 to 15, wherein

in the step of estimating stress, stress is estimated using a feature calculated from a signal other than the body-movement data.

(Supplementary Note 17)

A computer readable recording medium that includes recorded thereon a program including instructions that cause a computer to estimate stress of a measurement subject, the program including instructions that cause the computer to:

a step of acquiring body-movement data;

a step of storing body-movement data;

a step of calculating a stress-related body-movement feature from the stored body-movement data;

a step of calculating an estimated value of a going-out frequency from the stored body-movement data;

a step of correcting a correlation of the body-movement feature with stress using the body-movement feature and the going-out frequency;

a step of outputting the corrected body-movement feature; and

a step of estimating stress using the corrected body-movement feature.

(Supplementary Note 18)

The computer readable recording medium according to Supplementary note 17, wherein

in the step of calculating the estimated value of the going-out frequency,

the estimated value of the going-out frequency is calculated based on activity data of the measurement subject inferred from the body-movement data.

(Supplementary Note 19)

The computer readable recording medium according to Supplementary note 18, wherein

the activity data involves calculating a threshold for each individual measurement subject, wherein the calculation method of the threshold includes forming a histogram from numerical value data of a moving average of the change in body-movement data within a predetermined period and calculating a threshold for each activity state for each individual using the histogram, and a ratio of a specific activity is adopted as the activity data.

(Supplementary Note 20)

The computer readable recording medium according to Supplementary note 18, wherein

the activity data involves calculating a threshold that is common among measurement subjects, and the calculation method involves making a determination based on whether or not a moving average of the change in the body-movement data has exceeded a given threshold.

(Supplementary Note 21)

The computer readable recording medium according to Supplementary note 17, wherein

in the step of calculating the estimated value of the going-out frequency,

the estimated value of the going-out frequency is calculated based on the measurement subject's schedule data, office entry/exit record data, or transportation use record data, in place of the body-movement data.

(Supplementary Note 22)

The computer readable recording medium according to Supplementary note 17, wherein

in the step of calculating the estimated value of the going-out frequency,

the estimated value of the going-out frequency is calculated based on a report by the measurement subject himself/herself, in place of the body-movement data.

(Supplementary Note 23)

The computer readable recording medium according to any one of Supplementary notes 17 to 22, wherein

in the step of calculating the estimated value of the going-out frequency, the estimated value of the going-out frequency is calculated for a plurality of measurement subjects, and

in the step of estimating stress, the correlation of the body-movement feature with stress is corrected by performing multiplication with the reciprocal of a ratio of an average value for the measurement subject to an average value of the estimated values of the going-out frequencies of all measurement subjects.

(Supplementary Note 24)

The computer readable recording medium according to any one of Supplementary notes 17 to 23, wherein

in the step of estimating stress, stress is estimated using a feature calculated from a signal other than the body-movement data.

### REFERENCE SIGNS LIST

100 stress estimation apparatus

101 body-movement data acquisition unit

102 body-movement data storage unit

103 body-movement feature calculation unit

104 going-out frequency calculation unit

105 body-movement feature correction unit

106 corrected body-movement feature output unit

107 stress estimation unit

200 wearable terminal

300 measurement subject

502 portable terminal

504 Internet

600 computer

700 communication interface

801 body-movement data acquisition unit

802 body-movement data storage unit

803 body-movement feature calculation unit

804 body-movement feature storage unit

805 going-out frequency calculation unit

806 going-out frequency storage unit

807 body-movement feature correction unit

808 corrected body-movement feature storage unit

809 corrected body-movement feature output unit

901 stress estimation unit

902 stress estimation result storage unit

903 stress estimation result output unit

What is claimed is:

1. A stress estimation apparatus that estimates stress of a measurement subject, comprising:
a body-movement data acquisition unit that acquires body-movement data;
a body-movement data storage unit that stores body-movement data;
a body-movement feature calculation unit that calculates a stress-related body-movement feature from the stored body-movement data;
a going-out frequency calculation unit that calculates an estimated value of a going-out frequency from the stored body-movement data;
a body-movement feature correction unit that corrects a correlation of the body-movement feature with stress using the body-movement feature and the going-out frequency;
a corrected body-movement feature output unit that outputs the corrected body-movement feature; and
a stress estimation unit that estimates stress using the corrected body-movement feature.

2. The stress estimation apparatus according to claim 1, wherein
the going-out frequency calculation unit
calculates the estimated value of the going-out frequency based on activity data of the measurement subject inferred from the body-movement data.

3. The stress estimation apparatus according to claim 2, wherein
the activity data is data indicating the ratio of a specific activity among all activities performed by each individual measurement subject, and
the activity data is calculated by:
obtaining, for each individual measurement subject, a histogram indicating the frequency of each activity state based on a moving average obtained from a time-series change in the body-movement data;
further calculating a threshold for distinguishing each activity state using the obtained histogram; and
using the calculated threshold.

4. The stress estimation apparatus according to claim 2, wherein
the activity data is data indicating the ratio of a specific activity when a moving average obtained from a time-series change in the body-movement data is greater than or equal to a threshold that is common among measurement subjects.

5. The stress estimation apparatus according to claim 1, wherein
the going-out frequency calculation unit
calculates the estimated value of the going-out frequency based on the measurement subject's schedule data, office entry/exit record data, or transportation use record data, in place of the body-movement data.

6. The stress estimation apparatus according to claim 1, wherein
the going-out frequency calculation unit
calculates the estimated value of the going-out frequency based on a report by the measurement subject himself/herself, in place of the body-movement data.

7. The stress estimation apparatus according to claim 1, wherein
the going-out frequency calculation unit calculates the estimated value of the going-out frequency for a plurality of measurement subjects, and
the body-movement feature correction unit corrects the correlation of the body-movement feature with stress by performing multiplication with the reciprocal of a ratio of an average value for the measurement subject to an average value of the estimated values of the going-out frequencies of all measurement subjects.

8. The stress estimation apparatus according to claim 1, wherein
the stress estimation unit estimates stress using a feature calculated from a signal other than the body-movement data.

9. A stress estimation method for estimating stress of a measurement subject, comprising:
acquiring body-movement data;
storing body-movement data;
calculating a stress-related body-movement feature from the stored body-movement data;
calculating an estimated value of a going-out frequency from the stored body-movement data;
correcting a correlation of the body-movement feature with stress using the body-movement feature and the going-out frequency;
outputting the corrected body-movement feature; and
estimating stress using the corrected body-movement feature.

10. A non-transitory computer readable recording medium that includes recorded thereon a program including instructions that cause a computer to estimate stress of a measurement subject, the program including instructions that cause the computer to:
acquire body-movement data;
store body-movement data;
calculate a stress-related body-movement feature from the stored body-movement data;
calculate an estimated value of a going-out frequency from the stored body-movement data;
correct a correlation of the body-movement feature with stress using the body-movement feature and the going-out frequency;
output the corrected body-movement feature; and
estimate stress using the corrected body-movement feature.

11. The stress estimation method according to claim 9, wherein
when calculating the estimated value of the going-out frequency,
the estimated value of the going-out frequency is calculated based on activity data of the measurement subject inferred from the body-movement data.

12. The stress estimation method according to claim 11, wherein
the activity data involves calculating a threshold for each individual measurement subject, wherein the calculation method of the threshold includes forming a histogram from numerical value data of a moving average of the change in body-movement data within a predetermined period and calculating a threshold for each activity state for each individual using the histogram, and a ratio of a specific activity is adopted as the activity data.

13. The stress estimation method according to claim 11, wherein
the activity data involves calculating a threshold that is common among measurement subjects, and the calculation method involves making a determination based on whether or not a moving average of the change in the body-movement data has exceeded a given threshold.

14. The stress estimation method according to claim 9, wherein
when calculating the estimated value of the going-out frequency,
the estimated value of the going-out frequency is calculated based on the measurement subject's schedule data, office entry/exit record data, or transportation use record data, in place of the body-movement data.

15. The stress estimation method according to claim 9, wherein
when calculating the estimated value of the going-out frequency,
the estimated value of the going-out frequency is calculated based on a report by the measurement subject himself/herself, in place of the body-movement data.

16. The stress estimation method according to claim 9, wherein
when calculating the estimated value of the going-out frequency, the estimated value of the going-out frequency is calculated for a plurality of measurement subjects, and
when correcting the correlation, the correlation of the body-movement feature with stress is corrected by performing multiplication with the reciprocal of a ratio of an average value for the measurement subject to an average value of the estimated values of the going-out frequencies of all measurement subjects.

17. The stress estimation method according to claim 9, wherein
when estimating stress, stress is estimated using a feature calculated from a signal other than the body-movement data.
